(19) United States
(12) Patent Application Publication
(10) Pub. No.: US 2016/0349270 A1
(43) Pub. Date: Dec. 1, 2016

(54) FLIP (FLUORESCENCE IMMUNOPRECIPITATION) FOR HIGH-THROUGHPUT IMMUNOPRECIPITATION

(71) Applicant: THE JOHNS HOPKINS UNIVERSITY, Baltimore, MD (US)

(72) Inventors: Jef BOEKE, New York, NY (US); Paolo MITA, Brooklyn, NY (US)

(21) Appl. No.: 15/105,102

(22) PCT Filed: Dec. 16, 2014
(86) PCT No.: PCT/US14/70491

§ 371 Date: Jun. 16, 2016

Related U.S. Application Data
(60) Provisional application No. 61/916,329, filed on Dec. 16, 2013.

(51) Int. Cl. G01N 33/68; G01N 33/543; C12N 15/85; G01N 33/548

(52) U.S. Cl. CPC G01N 33/6854; G01N 33/548; G01N 33/54313; C12N 15/85

(57) ABSTRACT

This application describes an assay for immunoprecipitation that is quick, reliable, easy to perform, and that can be used in a high throughput fashion because it does not rely on western blotting analysis even if it can be included in a standard IP/WB procedure without affecting the output of the analysis. Because of these features the FLIP assay is ideal for the high-throughput screening of IP-grade antibodies. Here we present the basic concept of the invention and the application of the FLIP in high-throughput screening such as the quick identification of IP-proficient mouse monoclonal antibodies.

### BACKGROUND OF THE INVENTION

Field of the Invention

This application relates to an in vitro process of measuring and testing for the presence and functional recognition of target molecules using an antibody mixture. More specifically, this application is directed to an immunoprecipitation (IP) assay.

Description of the Background

The Immuno-Precipitation assay (IP) is a valuable assay that is applied in a variety of basic research as well as commercial applications such as targeted immunopurification, protein concentration, analysis of protein-protein interaction, identification/analysis of protein complexes, and analysis of protein/DNA interaction (Chromatin IP or ChIP). IP (and ChIP) is an inexpensive but highly informative technique that relies on the efficiency of a specific antibody (Ab) to selectively bind to the target peptide, protein, or protein complex of interest. By combining this binding reaction with a high molecular weight entity such as a bead or bacterial cell, or a meshwork of secondary antibodies, it is possible to pull down the antigen of interest in a microcentrifuge tube thereby separating the protein of interest and its binding partner(s) from all the other cellular components. Not all antibodies perform well in this particular application, however, because the antibody must “hang on” in the face of large hydrodynamic shear stress as the bead hurtles to the bottom of the centrifuge tube. Therefore, there is an increasing need for high-throughput assays for screening of antibodies capable to IP target proteins. Moreover the increasing application of monospecific antibodies in medicine such as for blocking antibodies or antibodies used in the treatment against viral infections (e.g. Ebola) underlies the necessity for fast and reliable methodologies for the screening of antibodies capable to selectively recognize a target protein. Moreover, because of the wide range of applications of the IP assay quick and high-throughput ways to determine the success of an IP are necessary. Up until now the standard procedure couples the IP assay to western blotting (IP/WB), a procedure that is not easily scalable to high-throughput analysis.

Immunoprecipitation assays take advantage of the binding in solution of an antibody to a specific target peptide, protein or protein complex. Beads conjugated to protein A (for rabbit antibodies), protein G (for mouse antibodies) or to protein A/G, or to certain bacterial cells displaying these proteins on their surface will bind the Ab-target complex, allowing the highly specific pull down of the target protein from a complex solution. The specificity is ensured by the highly selective interaction of the Ab to the target protein of interest. Washes of the beads coated with the Ab-target complex ensure the clean purification/concentration/isolation of the target protein of interest. In standard IP/WB technique (immunoprecipitation followed by western blotting analysis) the target protein or complex of interest is eluted from the beads and then visualized and analyzed through SDS-PAGE (SDS polyacrylamide gel electrophoresis) followed by western blotting. This existing method is time-consuming and relies on low-throughput gel electrophoresis and western blotting procedures.

### SUMMARY OF THE INVENTION

It is one object of the present invention to provide a method for the identification of antibodies able to recognize a target protein in its folded state. In one step of the method, a target protein operatively linked to a fluorescent protein is expressed in a host cell. In another step, crude or partially purified host cell lysate is collected. In a further step, the lysate is mixed with a primary antibody that binds to the target protein and beads coated with an affinity reagent. The affinity reagent binds to the primary antibody, creating a lysate-bead mixture that comprises the primary antibody bound to the target protein and the bead coated with the affinity reagent. The lysate-bead mixture is then centrifuged and the lysate-bead mixture is collected. In another step, the fluorescence of the lysate-bead mixture is measured using a manual fluorescence microscope, an automated microscopy system, or a fluorimeter.

Another object is to provide a recombinant expression vector for a FLIP assay. The vector comprises a regulated promoter; a tag nucleotide sequence expressing a fusion peptide comprising at least one antigen tag and a fluorescent protein tag; and a target nucleotide sequence multicloning site that allows any target protein to be expressed as a fusion protein with one or more of the antigen tags and fluorescent protein tags. The regulated promoter controls expression of the tagged antigen, or fluorescent protein tag can be removed through the use of recombinant methods.

A further object is to provide a kit for conducting FLIP assays. The kit comprises a vector for expression of a fluorescent protein and a target peptide, having a multicloning site for insertion of the target peptide; a cell line capable of expressing fluorescent protein and target peptide encoded in the vector; beads coated with an affinity reagent; and a set of buffers, tubes, or multiwell plates necessary to perform the FLIP assay.

A further object provides an instrument system for performing FLIP assays. The system comprises a microscope, an imaging system for measuring fluorescence, and a kit for conducting FLIP assays as described above.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1. Schematic of HuEV-A expression vector. Cloning of the gene of interest in the recombinant cassette will induce expression of a 3XFLAG, V5, YFP-tagged protein. The expression of the protein of interested is driven by a Tet-CMV promoter active only in the presence of doxycycline.

FIG. 2. Comparison between FLIP assay and conventional IP/WB assay. The arrow connecting the two procedures shows how the FLIP does not exclude the possibility of a conventional IP/WB analysis.

FIG. 3. Correlation between FLIP signal and initial amount of a YFP-tagged protein used for IP. The same samples used for FLIP analysis were then also processed for western blotting showed in the lower panel. IP with normal mouse IgG antibodies were used as control. Note that with this vector, the target protein always appears as two bands.

FIG. 4. FLIP assay performed using lysate of HuEV-A transfected cells plated in different size wells. One well was used for each IP (bar graph). The same samples used for FLIP were also processed for western blotting analysis (lower panel).

FIG. 5. FLIP analysis of 20 mouse antibodies produced by CDI laboratories. a) Reported in gray shaded cells are the antibodies that did not work for standard immunoprecipitation and that are shown to be also negative for FLIP assay. b) The FLIP signal is reported as the MEAN fluorescence from mAb IP minus the fluorescence from control IgG IP (mAb-IgG IP) obtained (ImageJ studio software was used to quantify the MEAN fluorescence of the collected pictures of the beads). The % IP was considered a good index for how well the Ab performed in the standard IP assay. The % IP is the amount of protein immuniprecipitated with a mAb compared to the total amount of expressed target-protein. The amount of total and immunoprecipitated protein was calculated from protein bands after western blotting analysis (Image studio 3.1 software was used to quantify the bands of a PVDF membrane scanned with a LiCoR Odyssey CLx scanner), displayed in a scatterplot format. 20 random mouse antibodies produced by CDI laboratories (Mayagüez, Puerto Rico) were screened for their ability to IP their respective target protein using FLIP assay integrated to standard IP/WB analysis. FLIP using the specific control IgG antibody subtracted from the MEAN fluorescence obtained from FLIP using the specific mAb. c) Correlation between the FLIP signal (X axis) and the % IP upon standard IP (Y axis).

### DETAILED DESCRIPTION OF THE INVENTION

The details of one or more implementations may be better understood by referring to the following description, claims, and accompanying drawings. The following description is of a particular embodiment of the invention, set out to enable one to practice an implementation of the invention, and is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

The term “antibody” or “Ab” refers to immunoglobulin molecules or fragments thereof, such as Fab, F(ab′)₂, and Fᵥ fragments, that are capable of binding an epitope on an antigen molecule. The term “antibody” is used in the broadest sense and specifically covers, but is not limited to, monoclonal antibodies (including full length monoclonal antibodies), polyclonal antibodies, monospecific and multispecific antibodies (e.g., bispecific antibodies). The term “antibody” also includes antibodies that comprise human immunoglobulin protein sequences only (i.e., “fully human” antibodies). A fully human antibody may contain murine carbohydrate chains if produced in a mouse, in a mouse cell, or in a hybridoma derived from a mouse cell. A fully human antibody may be generated in a human being, in a transgenic animal having human immunoglobulin germline sequences, by phage display or other molecular biological methods known to persons of ordinary skill in the art as described in U.S. Pat. No. 8,895,705. Also, recombinant immunoglobulins may also be made in transgenic mice.

The term “monospecific antibody” or “mAb” refers to an antibody that recognizes a single epitope on a target peptide. A “monoclonal antibody” refers to an antibody obtained from a population of substantially homogeneous antibodies, i.e., the individual antibodies comprising the population are identical except for possible naturally occurring mutations that may be present in minor amounts. The terms “monospecific” and “monoclonal” are used interchangeably herein. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method (see U.S. Pat. No. 8,895,705).

The term “nucleic acid” and “nucleic acid sequence” describe a nucleotide, oligonucleotide, polynucleotide, or any fragment thereof. These phrases also refer to DNA or RNA of any source and which may be single-stranded or double-stranded, or to any DNA-like or RNA-like material.

The phrase “regulated promoter” refers to a nucleic acid sequence operatively linked with a target nucleic acid sequence that encodes a protein or peptide of interest. In the present application the protein of interest consists of a fluorochrome conjugated to the protein. In some cases additional tag sequences may be included. The regulated promoter allows for control of expression of the protein of interest. In one exemplary embodiment, the regulated promoter is a Tet-On system, in which the peptide is expressed in the presence of doxycycline. Other regulated promoters may be used.

The phrase “operatively linked”, when describing the relationship between two nucleic acid regions, refers to an arrangement of the two sequences which allows them to function in their intended manner together. For example, a fluorescent sequence “operatively linked” to a target protein coding sequence results in a protein with a fluorescent tag. In some embodiments, the phrase refers to a promoter connected to a coding sequence such that transcription of that coding sequence is controlled and regulated by the promoter.

The term “tag” refers to a peptide molecule that is fused to a target protein and which can be recognized by known methods. Examples of tags include antigen tags such as FLAG and V5, which are recognized by commercially available or known Abs. The “tag” may also refer to fluorochromes, which can be detected through standard fluorescent microscopy methods.

The term “vector” is used to describe a nucleic acid molecule capable of expressing a desired peptide or protein construct in a given organism. A recombinant “vector” brings together various elements of the peptide or protein to be expressed, which provides the properties described in this application. In general, vectors used in recombinant DNA techniques are referred to as “plasmids” or double stranded DNA molecules that are capable of replicating and utilize the cellular machinery of their host to express their particular target peptide or protein.

There is an increasing need for mAbs that work for IP and Chromatin Immunoprecipitation (ChIP), which underlines the need for high-throughput IP procedures that are not available today. Also a fast and “high-throughputable” method for the selection of functional and specific antibodies is necessary for application in medicine. The fluorescence based IP (Fluorescence Immunoprecipitation or FLIP) described herein combines a comparable sensitivity to standard Western (immuno-) blotting and allows for high-throughput analysis not possible with canonical Western blotting analysis. The FLIP assay's efficiency is quickly and reliably measured, without the need to run time-consuming and low-throughput gel electrophoresis and western blotting procedures.

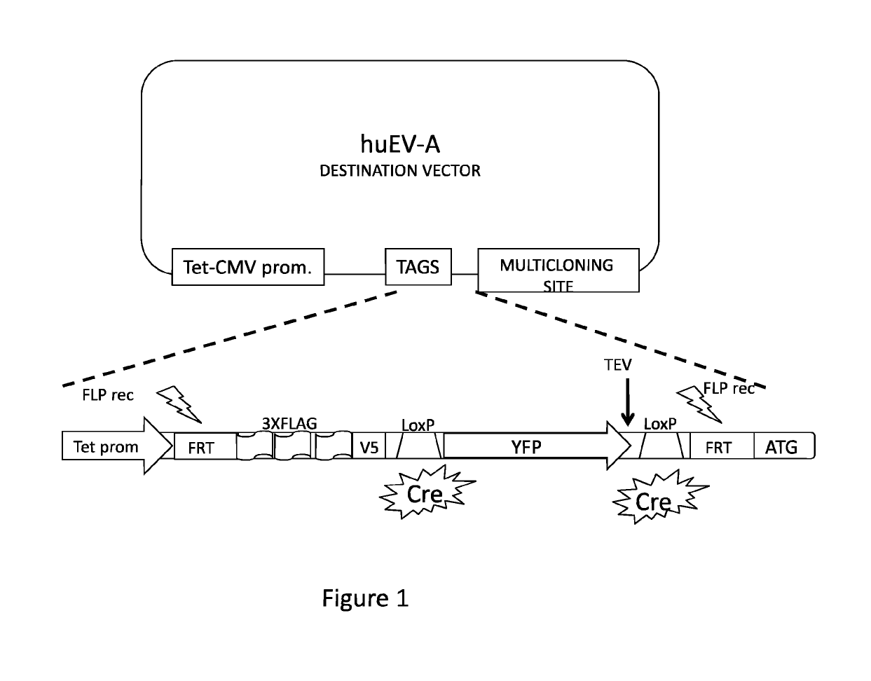

A recombinant expression vector is used to express a protein of interest, as graphically depicted in FIG. 1. The vector in its most basic form comprises a regulated promoter, a tag nucleotide sequence encoding a fluorescent protein tag, and a nucleotide sequence multicloning site that allows any ORF (open reading frame) to be expressed as a fusion protein with the selected tags. Once the target protein of interest is encoded in the vector and expressed in a host cell, the vector expresses a protein fused to a fluorescent tag, (e.g., a fluorochrome such as YFP [yellow fluorescent protein], GFP [green fluorescent protein], RFP [red fluorescence protein], CFP [cyan fluorescent protein], etc.). A person of ordinary skill would understand that other fluorochromes can be used and that the fluorescent tag is selected in such a way that it does not affect the normal folding of the target peptide. The vector also includes additional antigen tag nucleotide sequences for expressing a fusion protein comprising one or more known antigens for available antibodies.

In a preferred embodiment, the vector expresses the peptide of interest with at fluorescent tag, a triple FLAG tag, and a V5 tag. The presence of the FLAG and V5 tags allows for the use of commercial FLAG and V5 antibodies that can be used as positive controls for the assay. As described in more detail below, treating the lysate with anti-FLAG or anti-V5 antibodies allows a person of ordinary skill in the art to show that the target peptides are being expressed and to validate negative results. As with other known vectors, the vector used in a preferred embodiment includes a cloning cassette that allows any open reading frame (ORF) to be expressed as a fusion protein with the selected tags.

In one exemplary embodiment, a flexible mammalian, e.g., human, expression vector (HuEV) is used, which adds an N-terminus tag to the protein of interest (Sequence ID No. 1). It is contemplated that the tag may also be added to the C-terminus of the target protein of interest. The tag includes 3XFLAG, V5 and YFP. In some embodiments, a multicloning site is used that allows the introduction of the nucleic acid sequence for the protein of interest in the appropriate reading frame. The gene of interest can be easily cloned into the HuEV-A vector through the quick and highly efficient recombinant cloning system.

One advantage of the vector disclosed in this application is that it allows for the simple manipulation of the tags fused to the target protein. The tags can be removed by recombinant methods. For example, the length and composition of the tag added to the target protein can be controlled as desired with FLP or Cre recombinases. These enzymes allow one to express a) an untagged protein, b) a 3XFLAG-V5 tagged protein, or c) a 3XFLAG, V5 and YFP-tagged protein as shown on FIG. 1. However, any other vector enabling the joining of an ORF encoding a target protein of interest to a fluorescent tag at either the N- or C-terminus, or even tagged internally, may be used.

In one exemplary embodiment, a library of about 1500 transcription factors cloned into HuEV-A expression vector was developed to assess the effectiveness of hundreds of produced mAb using the FLIP assay described herein. Transcription factors are biologically important proteins for which commercial mAbs are not always available. This makes them a perfect and relevant vehicle to test the FLIP assay.

The FLIP assay is an optimal tool to screen for antibodies able to recognize and immunoprecipitate their target proteins. The FLIP assay described herein is a method for the identification of antibodies able to recognize a target protein in its folded state. In one step of the method, an expression vector comprising the target protein operatively linked to a fluorescent protein is provided. In another step, the fusion protein is expressed in the host cell, e.g., in mammalian cells for HuEV-A vectors. In a further step, the cells are lysed and crude or partially purified host cell, e.g., mammalian cell, lysate is collected. The lysate is then mixed with a primary antibody and beads coated with an affinity reagent recognizing the primary antibody (such as protein A/G coated agarose beads), creating a lysate-bead mixture. The lysate-bead mixture is centrifuged to separate the beads from other debris in the lysate. After washing of the beads with appropriate buffer, a sample of the centrifuged buffer-bead mixture is taken and the fluorescence of the beads is measured using a manual fluorescence microscope, an automated microscopy system, or a fluorimeter. The method can be performed in a multiwell plate format for high throughput screening as described in more detail below.

In the FLIP assay described herein the immuno-precipitated beads are washed and then directly visualized under a fluorescence microscope or automated microscopy system. If the YFP-tagged protein of interest has been successfully immuno-precipitated it will coat the beads that will fluoresce under light with an appropriate excitation wavelength. If the immunoprecipitation fails because the Ab did not recognize the protein of interest (or did not “hang on”), the beads will not be fluorescent and are not visualized under the fluorescence microscope. The FLIP assay is an easy, reliable and innovative assay that can be used in all the instances in which a standard IP/WB assay is applied to verify the efficient immuno-precipitation of a specific target protein expressed in the context of a fluorescent protein fusion such as YFP, GFP, RFP, etc. The FLIP assay takes advantage of the fluorescence signal emitted by the overexpressed target.

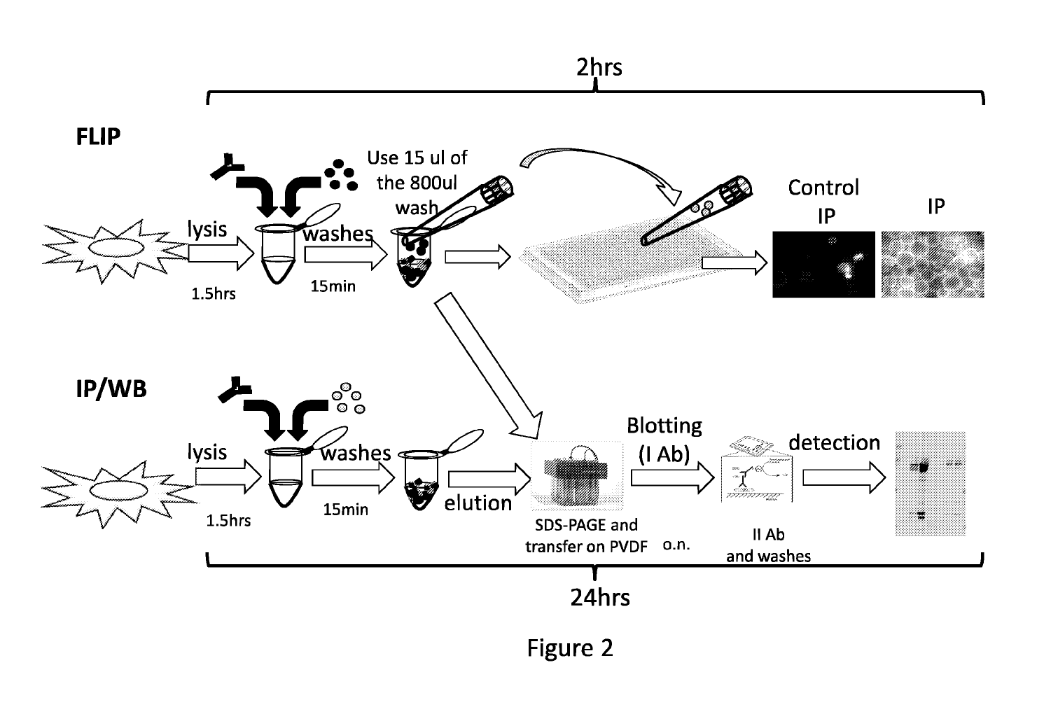

As shown on FIG. 2, quantification of the fluorescence signal of the beads against a control signal from a control IP performed using whole IgG from a nonimmunized mouse provides a reliable indication of the success of the IP and therefore of the binding of the Ab to the folded target protein. Therefore the FLIP assay is a much faster procedure compared to the standard IP/WB analysis because it is based on the direct observation of fluorescent target proteins coating the agarose beads. Moreover the FLIP assay can be integrated into a standard IP/WB analysis because only a minimal amount of beads is necessary for FLIP analysis. The left over beads can be processed for immunocomplex elution and follow-up analysis if necessary as shown in FIG. 2. The FLIP assay can be performed in a 384 well format. It can be subminiaturized further using appropriately designed plates with e.g. 1536 wells and automated using liquid handlers. The great advantage of the FLIP assay is the extremely short time of the analysis (the beads are directly analyzed after IP therefore eliminating any labor intensive analysis like western blotting) and its high-throughput capability.

The assay indeed utilizes just a small amount of the solution normally used for standard IP and it measures the fluorescence coating the beads upon successful immunoprecipitation. This feature of the FLIP has several advantages. It enables the integration of the FLIP assay into standard IP/WB protocols without disrupting the normal output of the procedure. It enables easy high-throughput optimization. The beads can be plated in 384 or even 1536 well plates for automated collection of pictures. The sensitivity of the assay and the small amount of beads necessary for the assay allows the miniaturization of the assay using lysate from a very small amount of cells expressing the YFP-tagged protein.

This unexpected breakthrough allows for the identification of positive results in an immunoprecitipation procedure without wasting time and resources in Western blots. A person of ordinary skill in the art would have expected that the small amount of beads used or the small amount of fluorescence-proteins coating the beads would not provide sufficient signal for identification of effective IPs. Here we show that the high correlation of the FLIP assay with standard IP makes the FLIP a perfect tool for large scale screening. We optimized and applied and develope the FLIP assay to the screening of IP positive mouse monoclonal antibodies potentially applicable to ChIP-seq analysis.

The FLIP assay can be used for many more applications. In one exemplary embodiment the FLIP assay can be used in screening of conditions or treatments that induce specific modifications such as phosphorylation or glycosylation of a protein of interest. In this context the protein of interest is overexpressed as a fluorochrome-tagged protein and immuno-precipitated with an antibody that specifically recognizes the modified proteins (e.g.: an antibody against a specific phosphorylated state of the target protein). Chemicals, siRNA and several different conditions can be screened for their ability to induce modification/phosphorylation of the target protein.

In another exemplary embodiment, the FLIP assay can be used in screening for proteins interacting with a protein of interest. This assay can be performed in two “library configurations.” In a first configuration, a single antibody and a library of cells expressing different GFP/YFP etc. fusion proteins can be used to identify proteins interacting with the endogenous protein of interest (specifically targeted by the chosen antibody). A specific IP-grade antibody against the (untagged) protein of interest can be used to immuno-precipitate overexpressed GFP/YFP tagged-proteins from cell lysates. If the protein of interest interacts with a specific GFP/YFP tagged-protein then the beads coated with the complexes including the protein of interest and the YFP/GFP-tagged protein will fluoresce under the microscope.

In another embodiment, one cell line expressing a single GFP/YFP etc. target-protein (bait) is used together with a library of antibodies that recognize proteins to be tested for their interaction with the target protein of interest. Specific IP-grade antibodies against the (untagged) proteins possibly interacting with the bait can be used to immunoprecipitate endogenous proteins from cell lysates. If a specific endogenous protein IPed by one of the Abs interacts with the GFP/YFP tagged-bait then the beads coated with the complexes including the interacting protein of interest and the YFP/GFP-tagged bait will fluoresce under the microscope.

The FLIP assay can be optimized as understood by a person of ordinary skill in the art. In one exemplary improvement of the FLIP assay, small agarose beads with uniform size are used. The beads may have a uniform size smaller than 25 micrometers. Commercially available agarose beads conjugated to proteinA and/or protein G have quite variable sizes. This variability introduces a higher variability in the analysis. Moreover smaller agarose beads will display a higher fluorescence because of a higher (fluorochrome amount)/(bead volume) ratio. Finally the smaller size of the beads ensure slower sedimentation of the beads on the bottom of the tube and therefore better efficiency of the washing as well as possibly better uniformity in the collection and plating of the beads in the plate before image acquisition. Methods exist in the literature for manufacture of uniform size agarose beads, e.g. Zhou et al J Colloid Interface Sci. 2007 311:118-27.

In another embodiment, agarose beads with uniform protein A/G coating are utilized. A uniform coating of protein A and/or G on the beads ensures a more uniform fluorescence signal for each bead and therefore a better analysis of the FLIP. The beads, in some embodiments, are coated with affinity reagents selected from the group consisting of protein A/G, protein A, protein G, Goat anti-mouse, nanobody, llamabody. In yet a further embodiment, colored agarose beads with no or low fluorescence are utilized. Few colored agarose beads are commercially available at this time and the available beads have a rather high fluorescence background because of the use of dyes with emission profiles partially overlapping with YFP or GFP fluorochromes, such as acid blue 9 (also known as Brilliant Blue FCF), and many other dyes. Low fluorescent background beads can be utilized for high through-put applications.

In yet a further embodiment, nonfluorescent magnetic beads can be used. Magnetic beads, similar to those sold under the DynaBeads® trademark, but that do not autofluoresce can also be used to employ magnetic separation technology, which would greatly facilitate the automation of the bead recovery and washing steps. The beads, in other embodiments, are made of magnetic material that allows magnetic separation and washing of beads from cell lysate.

A further embodiment image utilizes improved analysis software able to measure the fluorescence of the agarose beads as well as the area/volume of the beads themselves, which would improve the sensitivity of the FLIP assay because it would provide a way to normalize possible variability in number/size of beads from different pictures.

In a further embodiment, a kit for carrying out a FLIP assay comprises a vector; a cell line capable of expressing the vector; beads coated with an affinity reagent; and a set of buffers, tubes or multiwell plates necessary to perform the FLIP assay. The vector, as described above, comprises a regulated promoter and a nucleic acid sequence that codes a fluorochrome tag and a recombinant sequence that accepts an ORF of a target peptide. A user can insert a target gene utilizing the recombinant sequence, which results in the of a protein-fluorochrome complex. In some embodiments, the vector may also include other tags as described above. In one embodiment, the beads are agarose beads and the affinity reagent is protein A, protein G, or both.

A further embodiment relates to an instrument system comprising a microscope and an imaging system for measuring fluorescence. The microscope comprises an illuminator or light source, in some instances it may be a light emitting diode (LED) or a traditional light bulb. The microscope is configured to provide various contrast capabilities, such as epifluorescence or transmitted light (bright field and phase contrast). The microscope also includes various fluorescent channels and accommodates a number of fluorescent light cubes. The system further comprises a condenser with multiple positions. In one embodiment, the system comprises a monochrome camera, a color camera, or both, or a digital camera capable of capturing both color and monochrome pictures. The system further comprises software for measuring fluorescence of the subject samples viewed through the microscope. The system further comprises the kit capable of performing FLIP described above.

An automation station specialized for the hands-off processing of FLIP samples is also provide, which consists of a liquid handler, a multiwell plate handler and an imaging station or fluorimeter as understood by a person of ordinary skill in the art. A computer readable medium comprising instructions to analyze images obtained FLIP assay described above.

### Examples

The following is an exemplary FLIP protocol that allows for the high throughput experiments described herein. On day 1, Hela Tet-ON cells were plated in 6 well plates at a density of 0.3×10⁶ cells per well (1 wells will be used for a single IP). On day 2, the cells were transfected using Fugene-HD (Promega) and 0.75 μg of HuEV-A vector expressing the YFP tagged protein of interest and expression is induced adding 1 μg/ml doxycycline in the cell media. On day 3, the cells were harvested and lysed in lysis buffer (100 mM Tris-HCl pH 7.4, 150 mM NaCl, 25 mM NaF, 5004 ZnC12, 15% glycerol, 1% Triton X-100) supplemented with freshly added protease inhibitors (complete EDTA-free, Roche). Lysates are spun at 20000 rcf for 10 minutes at 4° C. and collected in a 96 deep-well plate. 5 μg of mAb or IgG control antibodies are added to the corresponding lysates. Ab-lysate solutions were incubated for 1 h at 4° C. under constant mixing on a Nutator® (TCS Scientific Corp.)(nutation). 50 μl of proteinA/G agarose beads (Sepharose4B beads coated with proteinA and proteinG; 40-165 μm diameter, cat. # sc-2003, Santa Cruz biotech.) were added to the mix and left nutating for an additional 30′ at 4° C. Beads were washed 3 times with 800 μl of lysis buffer. 15 μl of beads were collected during the last wash from each well. The wash/beads solutions were plated in a 384 black plate with clear bottom. A 12 channel multichannel pipette was used so that the control IgG IP beads were in wells adjacent to the corresponding mAb IP beads. Pictures of the fluorescence of the beads in the 384 well plate were collected using a BD pathway automated fluorescence microscope that collects and stitches together 4 pictures from each well of the 384 plate. After picture acquisition the fluorescence of the beads from each well was quantified using ImageJ. While pictures are being automatically recorded by the BD pathway fluorescence microscope the left over beads were pelleted and the immuno-complexes were eluted adding 50 μl of 1XLDS sample buffer to the beads.

The beads/sample buffer solution was then heated at 70° C. for 10 minutes and stored at −20° C. Standard SDS-PAGE was performed using the beads/sample buffer solution. After electrophoresis the proteins in the gel were transferred onto a PVDF (polyvinyl difluoride) membrane. Membranes were blocked and then incubated with a solution of primary antibody over night at 4° C.

On day 4, a standard IP/WB control was conducted. The membranes were washed several times and incubated for 1 hr at room temperature with a solution of secondary antibody conjugated with Dye800 fluorochrome. Membranes were washed again and then scanned using a LiCoR Odyssey CLx scanner. The bands of the considered target protein from the total lysate and IP samples were quantified using the Image Studio software.

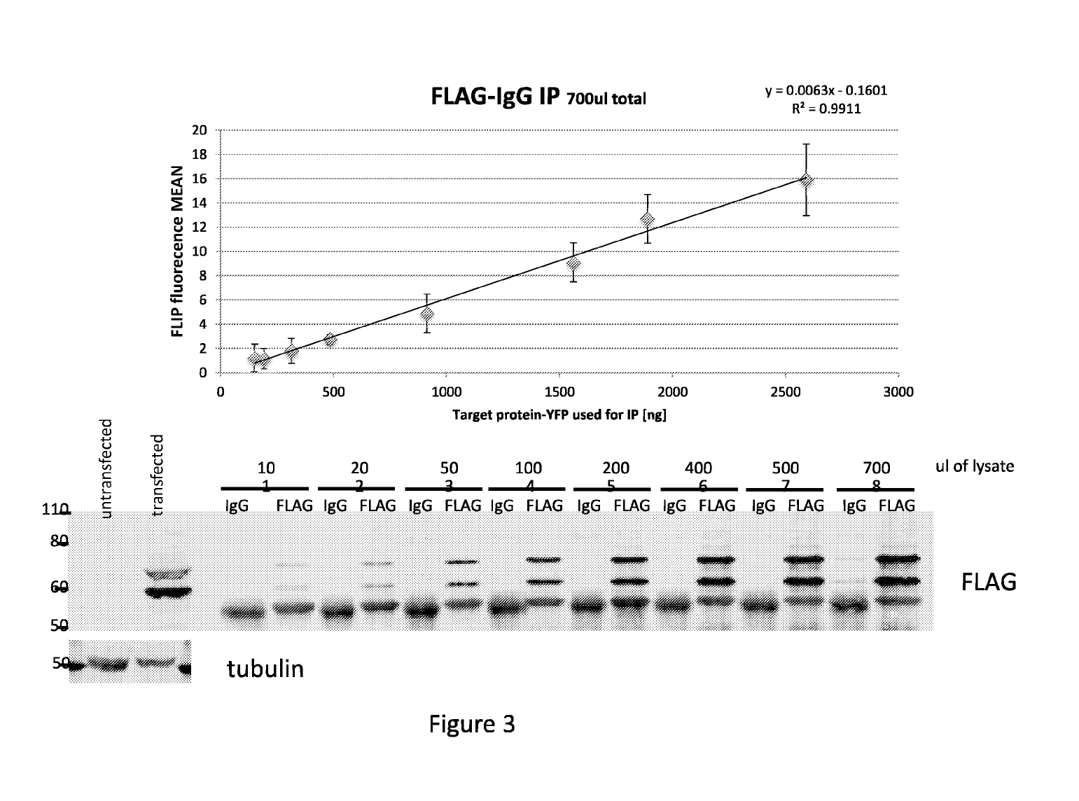

We investigated the correlation between the FLIP signal and the amount of target YFP-protein necessary for efficient FLIP. This analysis gave us an indication of the amount of over-expression necessary to have a reliable FLIP signal distinguishable from background. We performed FLIP using different amounts of protein overexpressed in HeLa Tet-ON cells (or other mammalian cell lines containing the Tet-ON Tet-OFF, or similar chemically regulated transcription system) transfected with standard procedures (Fugene-HD, Promega). The protein was expressed using the HuEV-A expression vector and treating the cells with 1 μg/ml doxycycline for 24 hrs to induce expression of the protein of interest. The YFP-tagged overexpressed protein was quantified measuring the fluorescence of the cell lysates using a spectrofluorimeter (excitation, 475 nm, emission, 527 nm) (FIG. 3, top panel). The nanograms of YFP present in the cell lysates then used for FLIP, was interpolated from a standard curve correlating the amount of purified recombinant YFP to the measured fluorescence.

The FLIP signal shows perfectly linear correlation with the amount of protein present in solution during immune-precipitation. Also, the same samples used for FLIP analysis were then processed for western blotting (FIG. 3, lower panel) following a protocol depicted in FIG. 2. The sensitivity of the FLIP assay is comparable to the western analysis and indeed a lower but still over-background FLIP signal was measured for the FLIP assay performed with the lowest amount of YFP-protein (amount 1). IP/WB performed with the same sample also shows a faint band with higher intensity than the background from the IgG control IP.

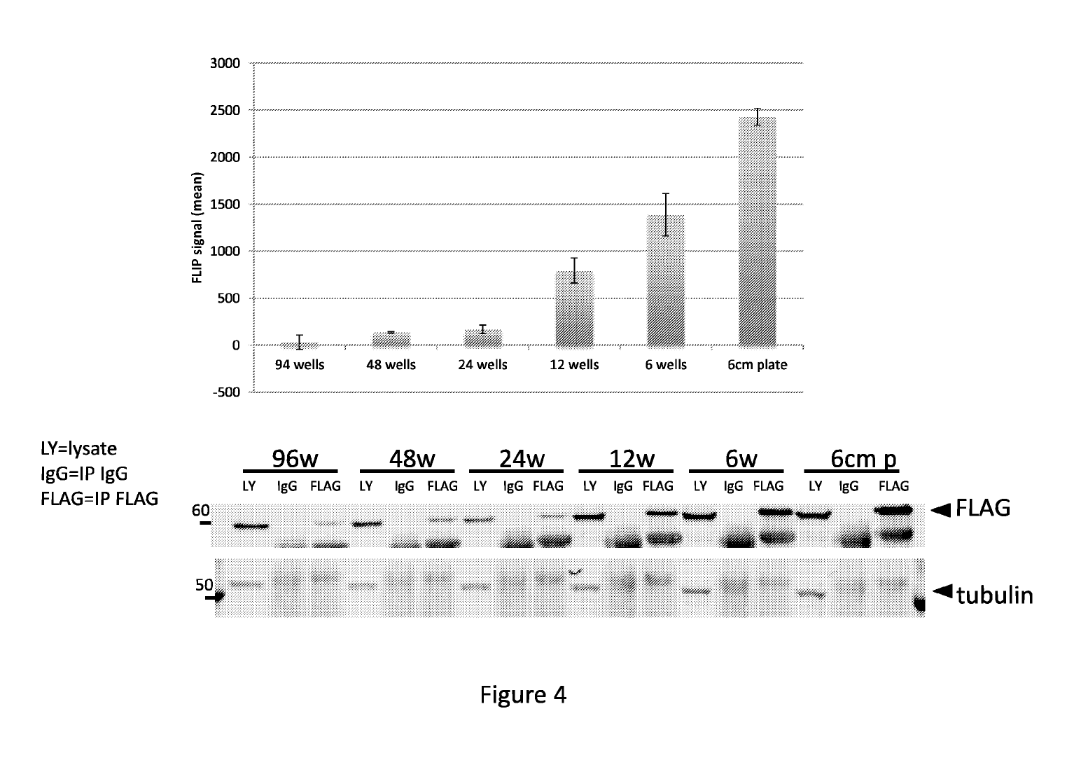

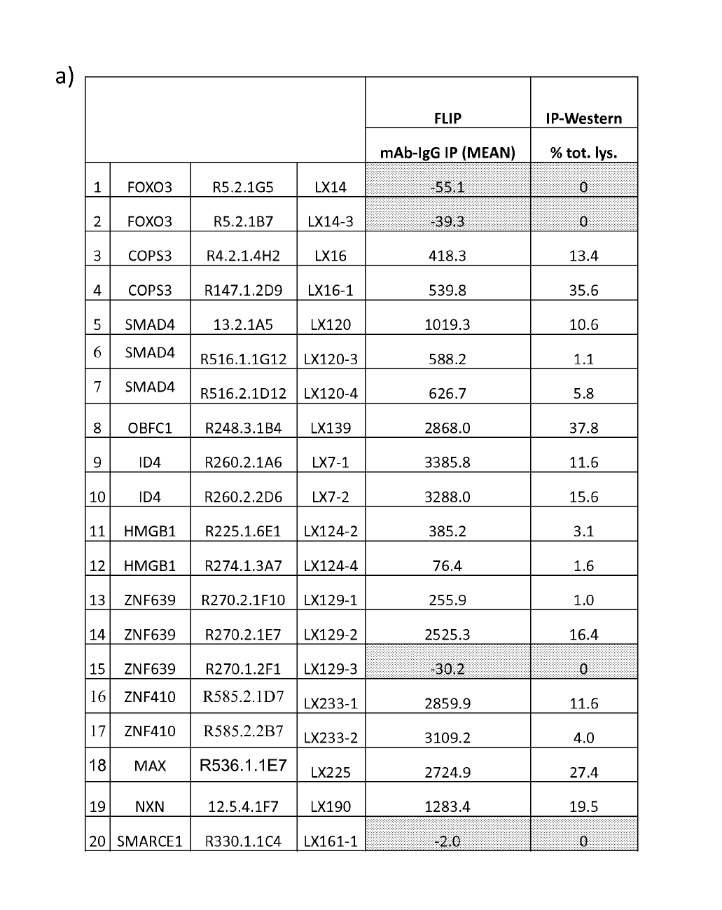

Experiments were conducted to determine how little starting lysate (and how few cells grown up to prepare the lysate) could be measured successfully. To show that the FLIP can be performed using lysates obtained from relatively few transfected cells we cultured HeLa Tet-on cells in 96, 48, 24, 12, 6 well plates and in 6 cm plates. We transfected cells plated in the different wells using the same HuEV-A construct expressing a YFP-tagged protein of interest as in the experiment shown in FIG. 3. Lysates were collected from each well and FLIP was performed using the lysate from one single well for each of the different size wells. (FIG. 4).

The experiment shows reliable reading over background starting from lysates collected from cells plated in 48 well plates. This demonstrates that the FLIP is an assay with comparable sensitivity to western blotting but with much higher high-throughput potential than western. Cells can be plated in 48 well plates and FLIP performed using just a small amount of beads. With optimization it may well be possible to subminiaturize this assay further. This small amount of beads (15 μl of beads solution from the washes performed in standard IP procedures) is plated in a 384 well plate and pictures of the fluorescence of the beads are collected as described above.

We then tested a high-throughput FLIP procedure described above to correlate the output from FLIP with the results from standard IP performed with the same beads that were used for FLIP (as in the scheme in FIG. 2). The FLIP fluorescence (FLAG-IgG IP mean fluorescence) was correlated to the amount of immune-precipitated antigen (% IP) compared to the initial amount of antigen present in the lysate before IP/FLIP assay. The % IP was calculated by quantification of the protein bands after western blotting analysis.

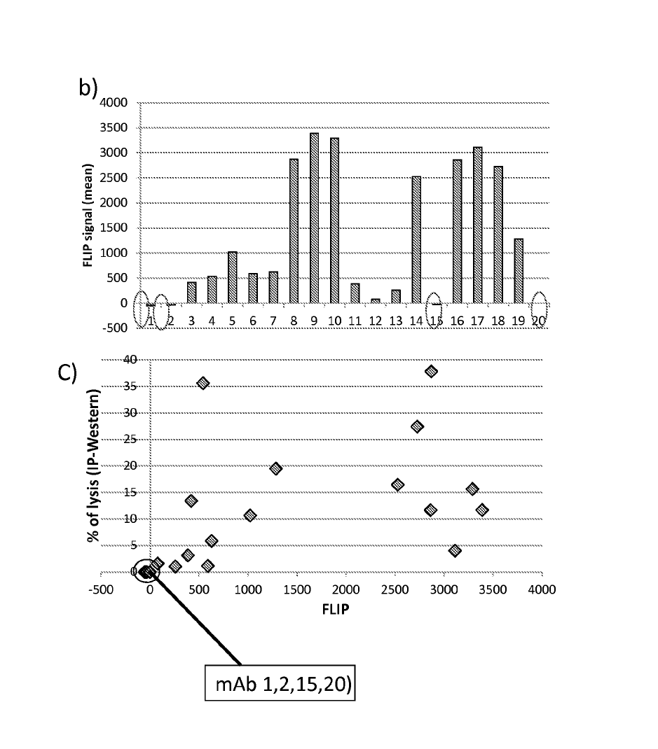

As reported in FIG. 5 the antibodies that did not work in standard immunoprecipitation assays (reported in red and with a % IP value equal to zero) did not pass the FLIP assay. The FLIP signal obtained using these antibodies, in fact, is lower than the signal obtained using a control mouse IgG antibody resulting to a negative value (FLIP=signal from mAb IP-signal from IgG control IP). This observation demonstrates that the FLIP assay is a reliable assay that can substitute for a standard IP/Western for the screening of antibodies positive for immunoprecipitation.

### INDUSTRIAL APPLICABILITY

The present invention is applicable to methods for identification of biological molecules. The invention discloses a method for conducting fluorescent immunoprecipitation assays and a vector for performing such assays. The method and devices described herein can be made and practiced in industry in the field of biotechnology.

1. A method for the identification of antibodies able to recognize a target protein in its folded state, comprising:
expressing a target protein operatively linked to a fluorescent protein in a host cell;
collecting a crude or partially purified host cell lysate;
mixing said lysate with a primary antibody that binds to said target protein and beads coated with an affinity reagent, wherein the affinity reagent binds to the primary antibody, creating a lysate-bead mixture that comprises the primary antibody bound to the target protein and the bead coated with the affinity reagent;
centrifuging said lysate-bead mixture;
collecting the lysate-bead mixture; and
measuring fluorescence of the lysate-bead mixture using a manual fluorescence microscope, an automated microscopy system, or a fluorimeter.

2. The method of claim 1, performed in a multiwell plate.

3. The method of claim 1, wherein the target protein operatively linked to a fluorescent tag is encoded in an expression vector.

4. The method of claim 3, wherein the vector comprises a regulated promoter.

5. The method of claim 1, wherein the host cell is a mammalian cell.

6. The method of claim 1, wherein the affinity reagent is selected from the group consisting of protein A, protein G, goat, anti-mouse, nanobodies, Uamabodies and other reagents capable of binding an antibody.

7. The method of claim 1, wherein the fluorescent protein is selected from the group consisting of a YFP—yellow fluorescent protein, GFP—green fluorescent protein, RFP—red fluorescent protein, and CFP—cyan fluorescent protein.

8. The method of claim 1, wherein the beads are agarose beads.

9. The method of claim 1, wherein the beads are of uniform size.

10. The method of claim 1, wherein the beads have a diameter of less than 25 micrometers.

11. A recombinant expression vector, comprising:
a regulated promoter;
a tag nucleotide sequence expressing a fusion peptide comprising at least one antigen tag and a fluorescent protein tag; and
a target nucleotide sequence multicloning site that allows any target protein to be expressed as a fusion protein with one or more of the antigen tags and fluorescent protein tags;
wherein the regulated promoter controls expression of the tagged antigen, or fluorescent protein tag can be removed through the use of recombinant methods.

12. The vector of claim 11, wherein the vector is a Human Expression Vector (HuEV) vector.

13. The vector of claim 11, wherein the at least one antigen tag is selected from the group consisting of a FLAG tag, a V5 tag, and other antigen tags.

14. The vector of claim 13, wherein the at least one antigen tag comprises a triple FLAG tag and a V5 tag.

15. The vector of claim 11, wherein the antigen tag and the fluorescent protein tag are attached to the N-terminus of the target protein.

16. The vector of claim 11, comprising a plurality of open reading frames from one or more organisms of interest forming a library of proteins of interest.

17. The vector of claim 11, wherein the fluorescent protein is selected from the group consisting of a YFP—yellow fluorescent protein, GFP—green fluorescent protein, RFP—red fluorescent protein, and CFP—cyan fluorescent protein.

18. A kit, comprising:
a vector for expression of a fluorescent protein and a target peptide, having a multicloning site for insertion of the target peptide;
a cell line capable of expressing fluorescent protein and target peptide encoded in the vector;
beads coated with an affinity reagent; and
a set of buffers, tubes, or multiwell plates necessary to perform the method of claim 1.

19. The kit of claim 18, wherein the vector is a Human Expression Vector (HuEV) vector.

20. The kit of claim 18, further comprising at least one antigen tag.

21. The kit of claim 20, wherein the at least one antigen tag is selected from the group consisting of a FLAG tag, a V5 tag, and other antigen tags.

22. The kit of claim 20, wherein the at least one antigen tag comprises a triple FLAG tag and a V5 tag.

23. The kit of claim 21, wherein the antigen tag and the fluorescent protein tag are attached to the N-terminus of the target protein.

24. The kit of claim 11, where the vector comprises a plurality of open reading frames from one or more organisms of interest forming a library of peptides of interest.

25. The vector of claim 18, wherein the fluorescent protein is selected from the group consisting of a YFP—yellow fluorescent protein, GFP—green fluorescent protein, RFP—red fluorescent protein, and CFP—cyan fluorescent protein.

26. An instrument system for performing FLIP assays, comprising:
a microscope, an imaging system for measuring fluorescence, and the kit of claim 18.

27. The system of claim 26, wherein the microscope comprises an illuminator or light source.

28. The system of claim 27, wherein the illuminator is selected from the group consisting of a light emitting diode (LED) or a traditional light bulb.

29. An automation station for the hands-off processing of FLIP samples, comprising a liquid handler, a multiwell plate handler, and an imaging station or fluorimeter.


SEQUENCE LISTING (1 sequence)

SEQ ID NO: 1 - DNA, 12383 bp, Artificial Sequence
  human cloning vector
cgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaaattgagcccgcagcctcccgcttcgctctctgctcctcctgttcgacagtcagccgcagaagttcttattctctagaaagtataggaacttcacaatggactacaaggacgacgacgacaaggattataaagatgatgatgataaagactataaggatgacgatgacaaaggcaagcccatccccaaccccctgctgggcctggacagcacctaccgttcgtataatgtatgctatacgaagttatccatggtgagcaagggcgaggagctgttcaccggggtggtgcccatcctggtcgagctggacggcgacgtaaacggccacaagttcagcgtgtccggtgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaactttaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagcacgtggaaaacctgtattttcagggcctggaagttctgttccaggggcccataacttcgtataatgtatgctatacgaagttatcgagaagttcctattctctagaaagtataggaacttcgagatcaacaagtttgtacaaaaaagctgaacgagaaacgtaaaatgatataaatatcaatatattaaattagattttgcataaaaaacagactacataatactgtaaaacacaacatatccagtcactatgtcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatggatccggtaccagctgctagcaagcttgctagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgcttttccaaaccaccctccttttttgcgccctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgaattcgtcgacctcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctggtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacgcgtccgc